(19) United States
(12) Patent Application Publication
(10) Pub. No.: US 2024/0342679 A1
(43) Pub. Date: Oct. 17, 2024

(54) ACTIVE OXYGEN SUPPLY DEVICE, DEVICE FOR CONDUCTING TREATMENT WITH ACTIVE OXYGEN, AND METHOD FOR CONDUCTING TREATMENT WITH ACTIVE OXYGEN

(71) Applicant: CANON KABUSHIKI KAISHA, Tokyo (JP)

(72) Inventors: SHOTA KANEKO, Tokyo (JP); KAZUHIRO YAMAUCHI, Shizuoka (JP); TAKUMI FURUKAWA, Shizuoka (JP); MASAKI OZAWA, Shizuoka (JP); KENJI TAKASHIMA, Kanagawa (JP); MOTOTERU GOTO, Shizuoka (JP)

(21) Appl. No.: 18/751,461

(22) Filed: Jun. 24, 2024

Related U.S. Application Data
(63) Continuation of application No. PCT/JP2022/048044, filed on Dec. 26, 2022.

(30) Foreign Application Priority Data
Dec. 28, 2021 (JP) 2021-215341

(51) Int. Cl. B01J 19/08; A61L 2/20; A61L 2/26; A61L 9/04; A61L 9/12; C01B 13/10; H05H 1/24

(52) U.S. Cl. CPC B01J 19/088; A61L 2/202; A61L 2/26; A61L 9/046; A61L 9/12; C01B 13/10; H05H 1/2406; A61L 2202/11; B01J 2219/0809; B01J 2219/0871; B01J 2219/0896; H05H 2245/15

(57) ABSTRACT

Provided is an active oxygen supply device capable of more efficiently supplying active oxygen to the surface of an object to be treated. An active oxygen supply device comprises a plasma actuator and a heater in a housing having at least one opening. The plasma actuator comprises a first electrode, a dielectric material and a second electrode, the first electrode is an exposed electrode provided on a first surface that is one of the surfaces of the dielectric material, when a voltage is applied between the first electrode and the second electrode, the plasma actuator generates the dielectric-barrier discharge oriented from the first electrode to the second electrode and induces a flow. The plasm actuator and the heater are disposed so that the induced flow containing active oxygen flows out of the housing through the opening.

### CROSS-REFERENCE TO RELATED APPLICATION

This is a continuation of International Application No. PCT/JP2022/048044, filed on Dec. 26, 2022, and designated the U.S., and claims priority from Japanese Patent Application No. 2021-215341 filed on Dec. 28, 2021, the entire contents of which are incorporated herein by reference.

### BACKGROUND OF THE INVENTION

### Field of the Invention

The present disclosure is directed to an active oxygen supply device, a treatment device using active oxygen, and a treatment method using active oxygen.

### Description of the Related Art

In recent years, ozone generators have become widely used for purposes such as cleaning the air inside rooms and cars, deodorizing refrigerators, and sterilizing hospitals. This is because ozone has strong oxidizing power. Also, if heat exceeding the activation energy is applied to ozone, the ozone will undergo thermal decomposition. At this time, the energy provided causes the ozone to become active oxygen, which has stronger oxidizing power, and is later decomposed into oxygen.

Japanese Patent Application Publication No. H03-109203 discloses an ozone generation device suitable for arrangement in a cold storage of a cold storage vehicle used for food delivery. The ozone generation device has a fan disposed on an inlet side of a casing, a heater disposed on an outlet side, and an electrical discharge portion of an ozonizer disposed between the heater and the fan. Also, Japanese Patent Application Publication No. H03-109203 discloses that in such an ozone generation device, air that is supplied from the inlet to the electrical discharge portion of the ozonizer by the action of the fan becomes ozonized air by passing through the electrical discharge portion, and the ozonized air is sent to the heater, heated by the heater, and thereafter is discharged from the outlet, and Japanese Patent Application Publication No. H03-109203 discloses that when the ozonized air passes through the heater, ozone is decomposed into air containing a large amount of oxygen molecules O₂ and nascent oxygen O, and is discharged.

### SUMMARY OF THE INVENTION

According to studies conducted by the present inventors, the disinfection and deodorization performance of the ozone generation device according to Japanese Patent Application Publication No. H03-109203 is limited, and in some cases the sterilization performance is comparable to that of a conventional sterilization method using only ozone. It is said that the disinfecting ability of active oxygen far exceeds that of ozone, so this study result was unexpected.

One aspect of the present disclosure aims to provide an active oxygen supply device that can more efficiently supply active oxygen to an object to be treated, a treatment device using active oxygen that can more efficiently treat the surface of an object to be treated with active oxygen, and a treatment method using active oxygen that can more efficiently treat the surface of an object to be treated with active oxygen.

At least one aspect of the present disclosure provides an active oxygen supply device comprising a plasma actuator and a heating device inside a housing having at least one opening,
wherein the plasma actuator is formed by stacking a first electrode, a dielectric material, and a second electrode in the stated order,
the first electrode is an exposed electrode provided on a first surface that is one surface of the dielectric material,
when a voltage is applied between the first electrode and the second electrode, the plasma actuator generates a dielectric-barrier discharge from the first electrode to the second electrode and expels an induced flow containing ozone in a first direction, which is one direction along a surface of the dielectric material from the first electrode,
the heating device heats the induced flow containing the ozone to generate active oxygen in the induced flow, and the induced flow becomes an induced flow containing the active oxygen, and
the plasma actuator and the heating device are arranged such that the induced flow containing the active oxygen flows out of the housing through the opening.

Further, at least one aspect of the present disclosure provides a treatment device using active oxygen, configured to treat a surface of an object to be treated with active oxygen, the treatment device comprising,
a plasma actuator and a heating device inside a housing having at least one opening,
wherein the plasma actuator is formed by stacking a first electrode, a dielectric material, and a second electrode in the stated order,
the first electrode is an exposed electrode provided on a first surface that is one surface of the dielectric material,
when a voltage is applied between the first electrode and the second electrode, the plasma actuator generates a dielectric-barrier discharge from the first electrode to the second electrode and expels an induced flow containing ozone in a first direction, which is one direction along a surface of the dielectric material from the first electrode,
the heating device heats the induced flow containing the ozone to generate active oxygen in the induced flow, and the induced flow becomes an induced flow containing the active oxygen, and
the plasma actuator and the heating device are arranged such that the induced flow containing the active oxygen flows out of the housing through the opening.

Furthermore, at least one aspect of the present disclosure provides a treatment method using active oxygen, for treating a surface of an object to be treated with active oxygen, the method comprising,
a step of preparing a treatment device using active oxygen,
wherein the treatment device using active oxygen includes a plasma actuator and a heating device in a housing having at least one opening,
the plasma actuator is formed by stacking a first electrode, a dielectric material, and a second electrode in the stated order,
the first electrode is an exposed electrode provided on a first surface that is one surface of the dielectric material,
when a voltage is applied between the first electrode and the second electrode, the plasma actuator generates a dielectric-barrier discharge from the first electrode to the second electrode and expels an induced flow containing ozone in a first direction, which is one direction along a surface of the dielectric material from the first electrode,
the heating device heats the induced flow containing the ozone to generate active oxygen in the induced flow, and the induced flow becomes an induced flow containing the active oxygen,
the plasma actuator and the heating device are arranged such that the induced flow containing the active oxygen flows out of the housing through the opening, and
the method further comprises:
a step of placing the prepared treatment device using active oxygen and the object to be treated at relative positions at which a surface of the object to be treated is exposed when the induced flow containing the active oxygen flows out through the opening; and
a step of allowing the induced flow containing the active oxygen to flow out through the opening to treat the surface of the object to be treated with active oxygen.

According to at least one aspect of the present disclosure, it is possible to obtain an active oxygen supply device that can more efficiently supply active oxygen to an object to be treated. Also, according to another aspect of the present disclosure, it is possible to obtain an active oxygen treatment device that can more efficiently treat the surface of an object to be treated with active oxygen. Furthermore, according to another aspect of the present disclosure, it is possible to obtain a treatment method using active oxygen that can more efficiently treat the surface of an object to be treated with active oxygen. Further features of the present invention will become apparent from the following description of exemplary embodiments with reference to the attached drawings.

### BRIEF DESCRIPTION OF THE DRAWINGS

FIGS. 1A and 1B are schematic cross-sectional views showing a configuration of an active oxygen supply device according to an embodiment of the present disclosure.

FIGS. 2A and 2B are schematic cross-sectional views showing a configuration of a plasma actuator according to one embodiment of the present disclosure.

FIG. 3 is an explanatory diagram of a plasma actuator according to an embodiment of the present disclosure.

FIG. 4 is a dimension explanatory diagram of an active oxygen supply device according to an embodiment of the present disclosure.

FIGS. 5A and 5B are schematic diagrams showing a relationship between a first electrode and a second electrode.

FIGS. 6A, 6B and 6C are schematic diagrams of a donut-shaped electrode.

### DESCRIPTION OF THE EMBODIMENTS

In the present disclosure, the descriptions “from XX to YY” and “XX to YY”, which express ranges of numerical values, mean numerical ranges including the lower limit and upper limit, which are the endpoints, unless otherwise specified. When numerical ranges are described in stages, the upper and lower limits of each numerical range can be combined as appropriate.

In the present disclosure, “treatment” of an object to be treated with active oxygen includes any treatment that can be achieved with active oxygen, such as surface modification (hydrophilization treatment) of the treatment surface of the object to be treated with active oxygen, disinfection, deodorization, and bleaching.

Also, “bacteria” as a target of “disinfection” according to the present disclosure refers to microorganisms, and the microorganisms include fungi, bacteria, unicellular algae, viruses, protozoa, and the like, as well as animal or plant cells (including stem cells, dedifferentiated cells, and differentiated cells), tissue cultures, fused cells obtained through genetic engineering (including hybridomas), dedifferentiated cells, and transformants (microorganisms). Examples of viruses include norovirus, rotavirus, influenza virus, adenovirus, coronavirus, measles virus, rubella virus, hepatitis virus, herpes virus, and HIV virus. Also, examples of bacteria include Staphylococcus, Escherichia coli, Salmonella enterica, Pseudomonas aeruginosa, Vibrio cholerae, Shigella, Bacillus anthracis, Mycobacterium tuberculosis, Clostridium botulinum, Clostridium tetani, Streptococcus, and the like. Furthermore, examples of fungi include Trichophyton, Aspergillus, Candida, and the like. Thus, in the present disclosure, “disinfection” also includes inactivation of viruses.

Furthermore, active oxygen in the present disclosure includes, for example, free radicals such as superoxide (·O₂—) and hydroxy radicals (·OH) generated by decomposition of ozone (O₃).

Hereinafter, embodiments for carrying out this disclosure will be specifically illustrated with reference to the drawings. However, the dimensions, materials, shapes, and relative arrangement of the components described in this mode are to be changed as appropriate depending on the configuration of the members to which the disclosure is applied and various conditions. That is, the scope of this disclosure is not intended to be limited to the following embodiments. Also, in the following description, components having the same functions are denoted by the same numbers in the drawings, and description thereof is omitted in some cases.

According to studies conducted by the inventors of the present invention, the reason why the ozone generation device according to Japanese Patent Application Publication No. H03-109203 has limited disinfecting and deodorizing abilities is inferred as follows.

Active oxygen is very unstable, and for example, the half-lives of ·O₂— and ·OH are at 10⁻⁶ seconds and 10⁻⁹ seconds respectively, which are extremely short, and thus it is thought that active oxygen is quickly converted into stable oxygen and water. In particular, in the ozone generator according to Japanese Patent Application Publication No. H03-109203, ozone sent by a fan is heated by a heater to excite the ozone and generate active oxygen. Indeed, it is thought that ozone is thermally decomposed in the surrounding area of the heater to generate nascent oxygen, that is, active oxygen. However, since the area near the heater is placed in the flow of air from a fan located upstream of the heater, it is thought that even if active oxygen is generated in the surrounding area of the heater, it will quickly be converted into oxygen and water in the turbulent flow of the air. Accordingly, the amount of active oxygen discharged from the discharge port of the ozone generation device to the outside of the device is small, and therefore it is thought that the disinfecting and deodorizing ability of the ozone generator according to Japanese Patent Application Publication No. H03-109203 is limited.

In view of this, the inventors of the present invention recognized that in treating an object to be treated using active oxygen, it is necessary to more actively place the object to be treated or the surface of the object to be treated in an active oxygen atmosphere. As a result of studies conducted by the inventors of the present invention based on this recognition, it was found that the active oxygen supply device according to the present disclosure described below enables the object to be treated to be more actively placed in an active oxygen atmosphere, and as a result, it is possible to more effectively treat the object to be treated.

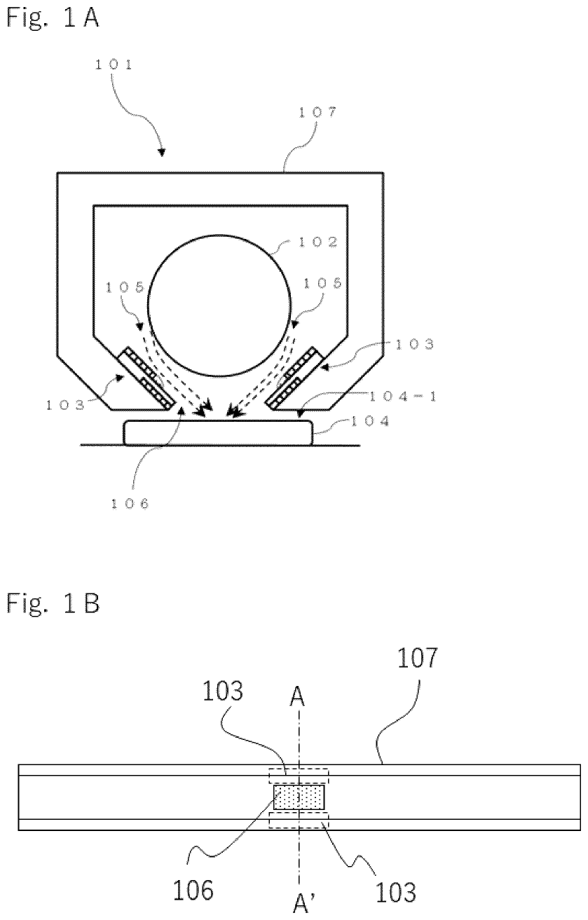

Hereinafter, an active oxygen supply device (treatment device using active oxygen) 101 according to an embodiment of the present disclosure will be described with reference to FIGS. 1A and 1B. An active oxygen supply device 101 according to an embodiment of the present disclosure includes a heating device 102 and plasma generation devices (plasma actuators) 103 inside a housing 107 having at least one opening 106.

The heating device 102 is arranged to be able to heat induced flows 105, and generates active oxygen in the induced flows 105. In FIG. 1A, reference numeral 104 indicates an object to be treated.

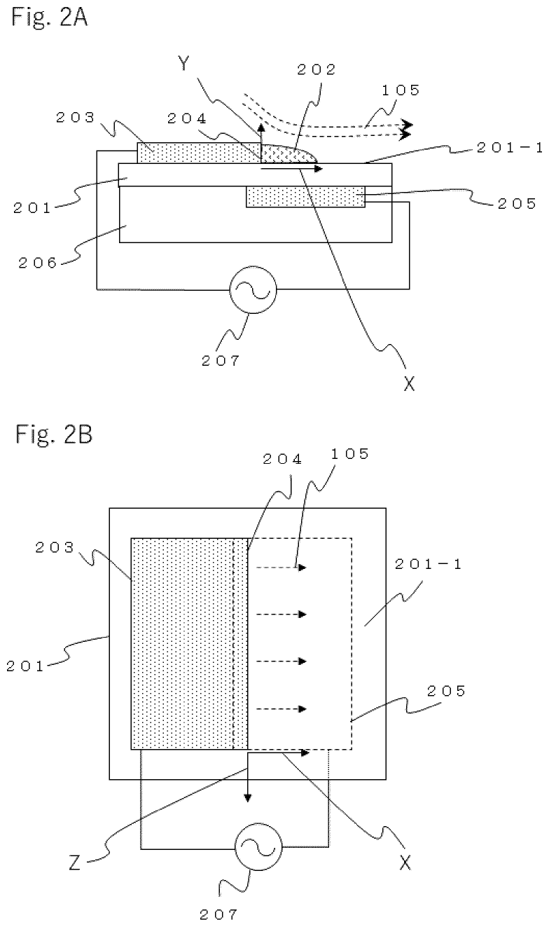

Also, a cross-sectional structure of an embodiment of the plasma actuator 103 is shown in FIG. 2A. The plasma actuator is a so-called dielectric-barrier discharge (DBD) plasma actuator (hereinafter referred to in some cases as “DBD-PA”) in which one surface of a dielectric material 201 (hereinafter also referred to as “first surface”) is provided with a first electrode 203, which is an exposed electrode with an exposed end surface, and another surface opposite to the first surface (hereinafter also referred to as “second surface”) is provided with a second electrode 205. In FIGS. 2A and 2B, reference numeral 206 indicates a dielectric substrate for embedding the second electrode 205 in the thickness direction of the plasma actuator so as not to generate an induced flow from the end surface of the second electrode. Also, a voltage can be applied to the first electrode and the second electrode by a power source 207.

In the plasma actuator 103, the first electrode 203 and the second electrode 205, which are arranged with the dielectric material 201 interposed therebetween, are arranged so as to be shifted diagonally opposite each other. By applying a voltage between these electrodes (between both electrodes) from the power source 207, a dielectric-barrier discharge is generated from the first electrode 203 toward the second electrode 205. Then, from an edge 204 of the first electrode 203 toward the direction in which the second electrode extends (arrow X direction in FIG. 2A), a jet-like flow is induced by plasma 202 along an exposed portion 201-1 (the portion that is not covered by the first electrode) of the first surface of the dielectric material 201.

At the same time, a suction flow of air is also generated from a space inside a container toward the electrodes. Electrons in the surface plasma 202 collide with oxygen molecules in the air, dissociating the oxygen molecules and producing oxygen atoms. The resulting oxygen atoms collide with undissociated oxygen molecules, generating ozone. Accordingly, an induced flow 105 containing highly-concentrated ozone is generated from the edge 204 of the first electrode 203 along the surface of the dielectric material 201 due to the action of the jet-like flow caused by the surface plasma 202 and the suction flow of air.

Also, the plasma actuator 103 and the heating device 102 are arranged so that the induced flow 105 containing active oxygen flows out of the housing 107 through the opening 106 and is supplied to a treatment surface 104-1 of the object to be treated 104.

That is, the plasma actuator is formed by stacking the first electrode 203, the dielectric material 201, and the second electrode 205 in the stated order, and the first electrode 203 is an exposed electrode provided on the first surface, which is one surface of the dielectric material 201. Then, by applying a voltage between the first electrode 203 and the second electrode 205, the plasma actuator generates a dielectric-barrier discharge from the first electrode 203 toward the second electrode 205, and expels an induced flow from the first electrode 203 in a first direction, which is one direction along the first surface of the dielectric material 201.

More specifically, a dielectric-barrier discharge is generated from one edge 204 of the first electrode 203 toward the second electrode 205, and an induced flow, which is a unidirectional jet, is expelled in a first direction (direction of the arrow X in FIG. 2A) along the first surface of the dielectric material 201 from the one edge 204 of the first electrode 203.

Also, in a cross-section taken in the thickness direction of the plasma actuator, the second electrode 205 extends in the expulsion direction (first direction) of the induced flow.

More specifically, for example, the plasma actuator has the dielectric material 201, and when a cross-section taken in the thickness direction of the plasma actuator is viewed, the first electrode 203 and the second electrode 205 are arranged in the thickness direction of the plasma actuator so as to be diagonally opposite each other with the dielectric material 201 interposed therebetween. The first electrode 203 is provided to cover a part of the first surface of the dielectric material 201, and the first surface of the dielectric material includes the exposed portion 201-1, which is not covered by the first electrode 203.

FIG. 2B is a diagram of the plasma actuator as viewed from the first surface side of the dielectric material. At least a portion of the exposed portion 201-1 and the second electrode 205 indicated by the broken line overlap with each other. Accordingly, the overlap is a region formed by the upper side, lower side, and right side of the broken line indicating the electrode 205 in FIG. 2B and the edge 204.

By applying a voltage between the first electrode and the second electrode, an induced flow containing ozone is generated along the exposed portion of the dielectric material overlapping with the second electrode 205 from the edge 204 on a side in the first direction of the first electrode 203 in the cross-section taken in the thickness direction (FIG. 2A).

Note that in the present disclosure, as shown in FIGS. 2A and 2B, the first direction along the surface of the dielectric material, which is the expulsion direction of the induced flow, is sometimes referred to as the X direction. Also, the axis that includes the X direction is called the X axis. For example, the X axis direction includes the X direction and the opposite direction to the X direction. The axis that is orthogonal to the X axis and is orthogonal to the first surface of the dielectric material is the Y axis. On the Y axis, the first electrode direction (e.g., a substantially vertically upward direction when the dielectric material is horizontal) in a view from the dielectric material is called the Y direction. Also, the direction that is orthogonal to the X direction and extends along the first surface of the dielectric material, that is, the axis orthogonal to the X and Y axes is called the Z axis.

The induced flow becomes, for example, a wall jet flow along the exposed portion 201-1, and it is easy to supply highly-concentrated ozone to a specific position. The length of the exposed portion 201-1 in the induced flow direction (i.e., the length from the edge 204 on a side in the first direction of the first electrode to the end of the first surface of the dielectric material) is not particularly limited, but is preferably 0.1 to 50 mm, more preferably 0.5 to 20 mm, and even more preferably 1.0 to 10 mm.

Accordingly, in the active oxygen supply device according to one aspect of the present disclosure, the induced flow 105 containing ozone from the plasma actuator 103 flows out of the housing 107 from the opening 106, and is supplied to the treatment surface 104-1 of the object to be treated 104. At the same time, the heating device 102 heats the induced flow 105 to generate active oxygen in the induced flow 105, and the induced flow becomes an induced flow containing active oxygen. As a result, active oxygen can be actively supplied to the region near the treatment surface 104-1, and specifically, for example, to a spatial region up to a height of about 1 mm from the treatment surface (hereinafter also referred to as “surface region”). For this reason, the generated active oxygen can be supplied to the surface of the object to be treated before the active oxygen is converted into oxygen and water. As a result, the treatment surface 104-1 of the object to be treated 104 is more reliably treated with active oxygen.

Also, since the induced flow 105 contains ozone in a high concentration, there is no need to increase the ozone concentration in the space from the heating device 102 to the surface region, and thus it is possible to prevent attenuation before reaching the surface region. As a result, ozone present in the surface region is efficiently decomposed into active oxygen by heat. Furthermore, as a result, active oxygen is generated on the processing surface 104-1 of the object to be treated or at a position very close to the treatment surface 104-1. As a result, the treatment surface 104-1 of the object to be treated 104 is placed in an active oxygen atmosphere generated in situ on the treatment surface, and the treatment surface is more reliably sterilized by active oxygen.

Also, since the induced flow 105 is generated by the plasma actuator 103, there is no need to provide another air flow generation device such as a blower fan. In particular, it is conceivable that the active oxygen contained in the induced flow generated by the plasma actuator according to the present disclosure can maintain an active state over a longer period of time than the commonly-stated lifespan of active oxygen (half life of ·O₂—: 10⁻⁶ seconds, half life of ·OH: 10⁻⁹ seconds). The reason for this is thought to be that active oxygen in the induced flow is protected in the organized flow of the induced flow, and collisions with other active species and molecules in the atmosphere are suppressed, making it difficult for deactivation due to reactions to occur.

For this reason, in the active oxygen supply device according to the present disclosure, it is preferable that, other than the plasma actuator, an air flow generation device such as a blower fan that generates an airflow inside the housing is not included. That is, it is preferable that the induced flow 105 itself, which is a jet-like flow generated from the plasma actuator 103, is supplied to the treatment surface 104-1 of the object to be treated 104. This suppresses disturbance of the organized flow of the induced flow, and suppresses active oxygen in the induced flow from colliding with the inner wall of the housing and from being deactivated by reaction with other active species. As a result, active oxygen can be efficiently supplied to the treatment surface 104-1 of the object to be treated 104.

### <Electrodes and Dielectric>

The materials constituting the first electrode and the second electrode are not particularly limited as long as they have good conductivity. For example, metals such as copper, aluminum, stainless steel, gold, silver, and platinum, plated or vapor-deposited materials, conductive carbon materials such as carbon black, graphite, and carbon nanotubes, and composite materials obtained by mixing these with resins or like can be used. The material constituting the first electrode and the material constituting the second electrode may be the same as or different from each other.

Among these, it is preferable that the material constituting the first electrode is aluminum, stainless steel, or silver, from the viewpoint of avoiding corrosion of the electrode and achieving uniform discharge. For the same reason, the material constituting the second electrode is also preferably aluminum, stainless steel, or silver.

Also, the shapes of the first electrode and the second electrode can be flat, wire-shaped, needle-shaped, or the like without particular limitation. Preferably, the first electrode has a flat shape. Also, preferably, the second electrode has a flat shape. When at least one of the first electrode and the second electrode has a flat shape, it is preferable that the aspect ratio (length of long side/length of short side) of the flat plate is 2 or more.

The dielectric material is not particularly limited as long as it is a material with high electrical insulation. For example, resins such as polyimide, polyester, fluororesin, silicone resin, acrylic resin, and phenol resin, glass, ceramics, and composite materials obtained by mixing these with resins or the like can be used. Among these, ceramics, glass, and silicone resin are preferably used as the dielectric material from the viewpoint of strength and insulation. In particular, since silicone resin is flexible, the degree of freedom in the shape of the plasma actuator can be increased.

### <Plasma Actuator>

The plasma actuator is not particularly limited as long as it is provided with a first electrode and a second electrode with a dielectric material interposed therebetween, and can generate an induced flow that is a unidirectional jet containing ozone by applying a voltage between the two electrodes.

In a plasma actuator, the shorter the shortest distance between the first electrode and the second electrode is, the more likely plasma is generated. For this reason, the film thickness of the dielectric material is preferably as thin as possible without causing electrical breakdown, and can be 10 μm to 1000 μm, and preferably 10 μm to 200 μm. Also, the shortest distance between the first electrode and the second electrode is preferably 200 μm or less. More preferably, it is 100 μm to 200 μm.

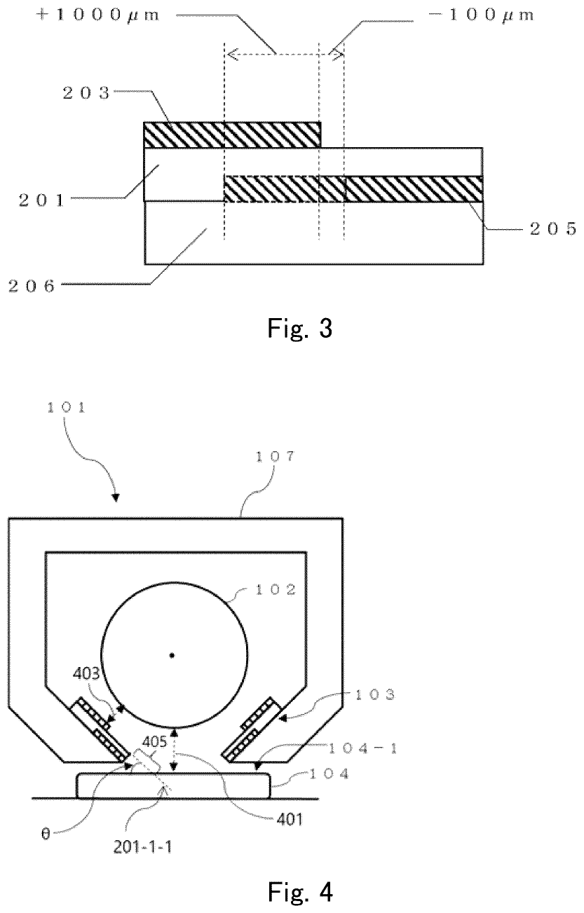

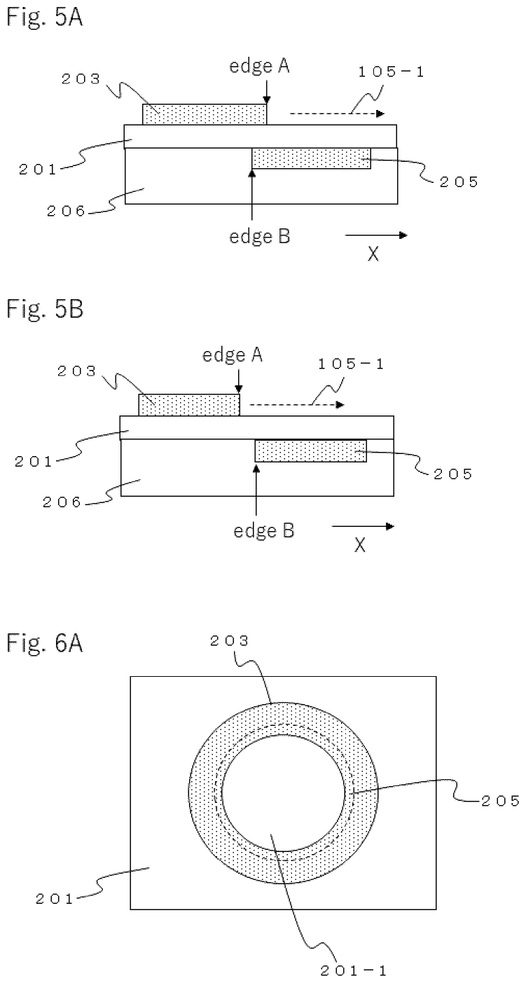

FIG. 3, FIG. 5A, and FIG. 5B are explanatory diagrams regarding the overlap between the first electrode 203 and the second electrode 205 of the plasma actuator, which is an ozone generator. FIG. 2 is a cross-sectional view of the plasma actuator.

The first electrode 203 and the second electrode 205 arranged diagonally opposite each other may be such that the edge of the first electrode is present at a formation portion of the second electrode with the dielectric material interposed therebetween, in a view through the plasma actuator from the first electrode (first surface) side. For example, the first electrode and the second electrode may be provided so as to overlap with each other in the Y-axis direction with the dielectric material interposed therebetween. In this case, it is preferable to prevent dielectric material breakdown during voltage application in the portion where the first electrode and the second electrode overlap with each other with the dielectric material interposed therebetween.

FIG. 5A shows a mode in which the first electrode and the second electrode overlap with each other (in the Y axis direction) with the dielectric material interposed therebetween. In the cross-section of the plasma actuator taken in the thickness direction, the edge of the first electrode on a side in a first direction is denoted as an edge A, and the edge of the second electrode on a side in a second direction (side in the direction opposite to the X direction), which is the direction opposite to the first direction, is denoted as an edge B. At this time, the edge B is preferably located in the second direction (in the direction opposite to the X direction) relative to the edge A.

Also, in a view through the plasma actuator from the first electrode side, when the edge of the first electrode on a side in a first direction is denoted as an edge A and the edge of the second electrode on a side in a second direction (side in the direction opposite to in the X direction), which is the direction opposite to the first direction, is denoted as an edge B, the edge B is located in the second direction (in the direction opposite to the X direction) relative to the edge A.

Due to the first electrode and the second electrode overlapping each other with the dielectric material interposed therebetween in this manner, stable plasma and induced flow can be generated.

Also, since the first electrode and the second electrode are arranged diagonally opposite each other with the dielectric material 201 interposed therebetween, the edge B is located in the first direction (X direction) relative to the edge opposite to the edge A of the first electrode. This makes it possible to suppress the generation of induced flow from the edge of the first electrode opposite to the edge A.

FIG. 5B shows a mode in which the first electrode and the second electrode do not overlap with each other (in the Y axis direction) with the dielectric material interposed therebetween. In the cross-section of the plasma actuator taken in the thickness direction, when the edge of the first electrode on a side in a first direction is denoted as an edge A, and the edge of the second electrode on a side in a second direction (side in the direction opposite to the X direction), which is the direction opposite to the first direction, is denoted as an edge B, the edge B is preferably located in the first direction (in the X direction) relative to the edge A.

Also, in a view through the plasma actuator from the first electrode side, when the edge of the first electrode on a side in a first direction is denoted as an edge A and the edge of the second electrode on a side in a second direction (side in the direction opposite to the X direction), which is the direction opposite to the first direction, is denoted as an edge B, the edge B is preferably located in the first direction (in the X direction) relative to the edge A.

In this way, when the first electrode and the second electrode do not overlap with each other with the dielectric material arranged therebetween, the voltage applied to the two electrodes is preferably relatively increased in order to compensate for the weakening of the electric field due to the shortest distance between the electrodes becoming relatively larger.

Also, in one preferable aspect, in a view through the plasma actuator from the first electrode side, when the edge of the first electrode on a side in a first direction is denoted as an edge A and the edge of the second electrode on a side in a second direction (side in the direction opposite to the X direction), which is the direction opposite to the first direction, is denoted as an edge B, the edge A and the edge B coincide with each other in the thickness direction (Y axis direction) of the dielectric material. Furthermore, in one preferable aspect, in a cross-section of the plasma actuator taken in the thickness direction, the edge A and the edge B coincide with each other in the thickness direction (Y axis direction) of the dielectric material. In this mode, for example, the edge A and the edge B face each other at the shortest distance with the dielectric material interposed therebetween, and the first electrode and the second electrode do not overlap with each other across the dielectric material, nor are they spaced apart from each other. As a result, the energy applied between both electrodes can be used more efficiently to generate the induced flow.

The overlap between the edge A of the first electrode and the edge B of the second electrode is preferably-100 μm to +1000 μm in the X axis direction in a view from above the cross-sectional view, assuming that the overlap length is positive, and it is more preferably 0 μm to +200 μm, and even more preferably 0 μm (FIG. 3). That is, if the edge B is located on the side in the direction opposite to the expulsion direction of the induced flow with respect to the edge A, then the interval in the direction along the surface of the dielectric material (X axis direction) between the edge A and the edge B is preferably-100 μm to +1000 μm, more preferably 0 μm to +200 μm, and even more preferably 0 μm. However, from the viewpoint of machining accuracy in plasma actuator production, it is difficult to always process the overlap to 0 μm over the Z axis direction. Thus, it is customary to provide a positive overlap according to the machining error.

There is no particular limitation on the thicknesses of the electrodes for both the first electrode and the second electrode, but the thicknesses can each be 10 μm to 1000 μm. If the thickness is 10 μm or more, the resistance becomes low and plasma is easily generated. If the thickness is 1000 μm or less, electric field concentration tends to occur, making it easier to generate plasma.

The widths of the electrodes for both the first electrode and the second electrode are not particularly limited, but can each be 1000 μm or more.

Although the shape of the electrode is not particularly limited, it is preferably a rectangular shape, such as a rectangle or a square. Due to being rectangular, a uniform induced flow can be generated.

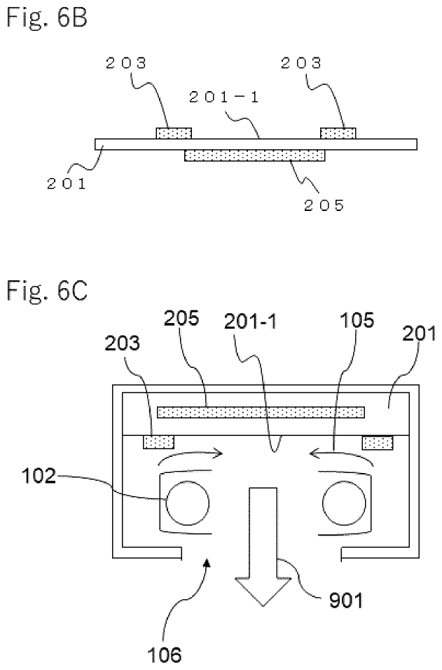

Also, as shown in FIG. 6A (perspective view from the first surface side of the dielectric material) and FIG. 6B (cross-sectional view taken in the thickness direction of the plasma actuator), the first electrode may be donut-shaped, and the second electrode may be circular or donut-shaped. Even with this type of electrode, when viewing the cross-section taken in the thickness direction, the first electrode 203 and the second electrode 205 are arranged diagonally opposite each other in the thickness direction of the plasma actuator with the dielectric material 201 interposed therebetween. The first electrode 203 is provided to cover a part of the first surface of the dielectric material 201, and the first surface has an exposed portion 201-1 that is not covered by the first electrode 203. Furthermore, in a view through the plasma actuator from the first surface side (FIG. 6A), there is an overlap between at least a part of the exposed portion 201-1 of the dielectric material and the second electrode 205 (the doughnut-shaped hole part of the first electrode).

Even in the case of such a donut-shaped electrode DBD-PA, by applying a voltage between the first electrode and the second electrode, a dielectric-barrier discharge from the first electrode to the second electrode is generated, and an induced flow is expelled from the first electrode in one direction along the surface of the dielectric material. The ejected induced flow collides near the center of the electrode and becomes an axisymmetric jet (three-dimensional wall normal jet) that ejects upward in FIG. 6B.

Also, an example of an active oxygen supply device using such a donut-shaped electrode DBD-PA is shown in FIG. 6C. In the active oxygen supply device shown in FIG. 6C, an induced flow 105 containing ozone is ejected from the inner edge of the donut-shaped first electrode, toward the first direction along the surface of the dielectric material 201, that is, toward the center. Then, due to colliding with the center of the first electrode, the induced flow 105 causes an axisymmetric jet 901 containing ozone to flow in a direction orthogonal to the surface 201-1 of the dielectric material 201 (vertically downward in FIG. 6C). Then, by heating this axisymmetric jet 901 with the heater 102, ozone in the axisymmetric jet is decomposed into active oxygen, and an axisymmetric jet containing active oxygen flows out of the housing 107 through the opening 106. An object to be treated arranged near the opening 106 is treated with the active oxygen in the axisymmetric jet 901 flowing out from the opening.

Note that, as shown in FIG. 6C, the active oxygen treatment device according to the present disclosure may be arranged in the housing such that the heating device does not directly heat the object to be treated 104 placed at the opening of the active oxygen treatment device. In such an active oxygen supply device, unlike the case where the active oxygen treatment device shown in FIG. 1A is used, active oxygen is unlikely to be generated in situ on the surface of the object to be treated 104. However, due to the induced flow containing active oxygen flowing out from the opening of the housing, active oxygen can be actively supplied to the object to be treated 104, and the object to be treated 104 can be reliably treated.

Also, the plasma actuator may be a so-called three-electrode plasma actuator in which a third electrode is further provided on the first surface of the dielectric material 201, downstream in the expulsion direction of the induced flow from the first electrode. In this case, for example, an AC voltage can be applied by using the first electrode as an AC electrode, and a DC voltage can be applied by using the third electrode as a DC electrode. A sliding discharge can also be generated by applying a negative DC voltage to the DC electrode.

Also, if the edge of the second electrode is exposed, plasma is generated also from the edge of the second electrode, whereby an induced flow in the opposite direction to the induced flow 105 originating from the first electrode can be generated. In the active oxygen supply device according to this aspect, it is preferable that the ozone concentration in the internal space of the active oxygen supply device other than the surface region of the object to be treated is kept as low as possible. Also, it is preferable not to generate a flow of gas in the container that would disturb the flow of the induced flow 105. For this reason, it is preferable not to generate an induced flow originating from the second electrode.

In view of this, in order to prevent plasma from being generated from the second electrode 205, the second electrode 205 is preferably an embedded electrode. For example, as shown in FIGS. 2 and 3, the second electrode may be covered with a dielectric material as in the dielectric substrate 206, or may be embedded in the dielectric material 201.

The second electrode need only be embedded to an extent according to which it is possible to prevent the generation of plasma from the edge of the second electrode, and for example, a part of the surface of the second electrode may be exposed, and the exposed surface of the second substrate and the dielectric substrate 206 or dielectric material 201 may form the same plane. Preferably, the edge of the second electrode is covered by the dielectric substrate 206 or the dielectric material 201.

Accordingly, for example, the plasma actuator is preferably an SDBD (single dielectric-barrier discharge) plasma actuator.

In the plasma actuator, it is preferable that no induced flow is generated from an edge other than edge A of the first electrode defined as described above. For this reason, edges other than the edge A may be covered with a dielectric material. As a result, even if the first electrode and the second electrode overlap with each other in the Y axis direction, a unidirectional jet can be generated. Also, the shape of the electrode may be controlled so that no induced flow is generated from edges other than the edge A in relation to the second electrode. For example, if the electrode is rectangular, the length of the electrode in the Z axis direction (direction orthogonal to the expulsion direction of the induced flow from edge A) may be the same for the first electrode and the second electrode, or may be made longer for the first electrode. Such an embodiment makes it easier to actively supply the induced flow to the object to be treated.

The induced flow 105 containing highly-concentrated ozone flows in a jet-like flow direction due to the surface plasma along the exposed portion 201-1 of the first surface of the dielectric material 201 from the edge 204 of the first electrode 203, that is, in the direction along the exposed portion 201-1 of the first surface of the dielectric material from the edge 204 of the first electrode 203. This induced flow is a flow of gas containing highly-concentrated ozone and has a velocity of several m/s to several tens of m/s.

The voltage applied between the first electrode 203 and the second electrode 205 of the plasma actuator is not particularly limited as long as it can generate plasma in the plasma actuator. Also, although a DC voltage or an AC voltage may be used, an AC voltage is preferable. Also, in a preferable aspect, the voltage is a pulse voltage.

Furthermore, the amplitude and frequency of the voltage can be set as appropriate in order to adjust the flow rate of the induced flow and the ozone concentration in the induced flow. In this case, the amplitude and frequency of the voltage may be selected as appropriate from the viewpoint of generating, in the induced flow, an ozone concentration that is necessary to generate an effective active oxygen concentration or effective active oxygen amount corresponding to the purpose of treatment, from the viewpoint of supplying the generated active oxygen to the surface region of the object to be treated while maintaining the active oxygen concentration or the effective active oxygen amount corresponding to the purpose of the treatment.

For example, the amplitude of the voltage can be between 1 kV and 100 kV. Furthermore, the frequency of the voltage can be preferably 1 kHz or more, and more preferably 10 kHz to 100 kHz.

If the voltage is an AC voltage, the waveform of the AC voltage is not particularly limited, and a sine wave, a square wave, a triangular wave, or the like can be used, but a rectangular wave is preferable from the viewpoint of the speed at which the voltage rises.

Although the duty ratio of the voltage can also be selected as appropriate, it is preferable that the voltage rises quickly. Preferably, the voltage is applied so that the rise of the voltage from the bottom to the peak of the wavelength amplitude is 10,000,000 V/sec or more.

Note that the value obtained by dividing the amplitude of the voltage applied between the first electrode 203 and the second electrode 205 by the film thickness of the dielectric material 201 (voltage/film thickness) is preferably 10 kV/mm or more.

### <Heating Device>

The heating device 102 is not particularly limited as long as it can provide thermal energy that can excite ozone in the induced flow and generate active oxygen. Thermal decomposition of ozone starts at about 100° C., and therefore a device that can heat the induced flow to about 120° C. is preferable. On the other hand, in order to suppress the effects of heat on the object to be treated, such as melting and decomposition of the object to be treated, the temperature is preferably 200° C. or less. It is preferably 100 to 140° C., and more preferably 110 to 130° C.

The heating device is not particularly limited, and includes, for example, a device including a heat source (heat supply means) that supplies heat. Specific examples include ceramic heaters, cartridge heaters, sheathed heaters, electric heaters, and oil heaters. In the case of a device including a metal heating element, the heating element is preferably made of a material with excellent oxidation resistance, such as a nichrome alloy or tungsten. A cartridge heater is preferred.

### <Arrangement of Plasma Actuator, Heating Device, and Object to be Treated>

In the active oxygen supply device 101, the position of the plasma actuator 103 that generates an induced flow containing ozone is not particularly limited, as long as it is arranged such that the induced flow 105 flows out of the housing through the opening due to the heat from the heating device 102 while the effective active oxygen concentration or the effective active oxygen amount corresponding to the purpose of the treatment is maintained.

For example, the plasma actuator and the heating device may be arranged such that the induced flow 105 containing active oxygen generated by heat is supplied to the surface of the object to be treated over the shortest distance.

Also, for example, the object to be treated may be arranged such that the treatment surface 104-1 of the object to be treated is included on an extended line in a direction along the first surface (the exposed portion 201-1 of the first surface) of the dielectric material from the edge 204 on the side of the first electrode 203 of the plasma actuator in the first direction. For example, it is preferable that the extension line is in contact with the treatment surface 104-1.

Also, an extension line in the direction along the first surface of the dielectric material (the same as the X direction) from the edge on the side of the first electrode 203 of the plasma actuator in the first direction is preferably directed toward the opening. This makes it easier for the induced flow to flow out of the housing through the opening.

Furthermore, when the opening of the active oxygen supply device is directed vertically downward, the narrow angle formed by a horizontal plane (plane orthogonal to the vertical direction) and an extension line 201-1-1 extending in the direction along the exposed portion 201-1 of the first surface of the dielectric material from the edge of the first electrode of the plasma actuator is 0 (hereinafter also referred to as the plasma actuator incidence angle or PA incidence angle; see FIG. 4). The narrow angle θ is not particularly limited as long as it is an angle at which an induced flow can be actively supplied to the surface region of the object to be treated while maintaining the effective active oxygen or the amount of effective active oxygen corresponding to the purpose of the treatment, or an angle at which treatment can be performed with the active oxygen, and the narrow angle θ is preferably 0° to 90°, and more preferably 30° to 70°.

By arranging the plasma actuator and the heating device as described above, an induced flow containing active oxygen and having a certain flow rate can be locally supplied to a region near the surface of the object to be treated, or treatment can be performed with the active oxygen. Also, the induced flow that has flowed out from the opening flows along the treatment surface of the object to be treated, and parts of the treatment surface of the object to be treated other than the portion facing the opening are also exposed to the induced flow containing active oxygen. This allows a wider range of the treatment surface 104-1 to be treated with active oxygen.

Also, the plasma actuator is preferably arranged so that the treatment surface 104-1 of the object to be treated is included on the extension line in the first direction (the expulsion direction of the induced flow). When the opening of the active oxygen supply device is directed vertically downward, the narrow angle formed by the first direction (the expulsion direction of the induced flow) and the horizontal plane (the plane orthogonal to the vertical direction) is 0′. The angle θ′ is preferably 0° to 90°, and more preferably 30° to 70°.

The heating device 102 is not particularly limited as long as it heats the induced flow containing ozone, generates active oxygen in the induced flow, and is arranged such that treatment on the surface of the object to be treated is possible while maintaining the effective active oxygen concentration or the effective active oxygen amount corresponding to the purpose of the treatment.

As described above, an induced flow containing ozone is actively supplied to a region near the surface of the object to be treated. Also, active oxygen can be generated in the induced flow by heating the induced flow. For this reason, by heating the induced flow, ozone is excited, and the induced flow in which active oxygen has been generated can be actively supplied to the surface of the object to be treated, and it is possible to significantly increase the active oxygen concentration or the active oxygen amount on the surface of the object to be treated.

The relative position of the heating device and the plasma actuator is not particularly limited, as long as the heating device and the plasma actuator are arranged such that active oxygen is generated in the induced flow and treatment on the surface of the object to be treated is possible while maintaining the effective active oxygen concentration or the effective active oxygen amount corresponding to the purpose of the treatment.

As for the distance between the heating device and the plasma actuator, the intensity and the position with respect to the plasma actuator need only be set such that the induced flow is heated, active oxygen is generated in the induced flow, and the induced flow containing an effective amount of active oxygen corresponding to the purpose of the treatment flows out of the housing through the opening and is supplied to the object to be treated. As an example of the arrangement position of the heating device with respect to the plasma actuator, for example, the distance between the dielectric material of the plasma actuator and the surface facing the heating device is preferably 10 mm or less, and more preferably 4 mm or less. However, it is not necessary to place the plasma actuator within about 10 mm from the heating device, and the distance between the heating device and the plasma actuator is not particularly limited as long as the active oxygen in the induced flow can reach the effective concentration corresponding to the purpose of the treatment in relation to the generated heat and the like.

Also, in a preferred aspect, at least one of the heating device and the plasma actuator is provided with a moving means and at least one of the heating device and the plasma actuator is movable such that uniform heating can be achieved.

As for the relative position of the active oxygen supply device and the object to be treated, it is sufficient that at least one of the active oxygen supply device and the object to be treated is arranged such that active oxygen is generated in the induced flow and the surface of the object to be treated is exposed to the induced flow maintained at the effective active oxygen concentration or the effective active oxygen amount corresponding to the purpose of the treatment.

Also, the heating device may be arranged at a position where the surface of the object to be treated can be heated, or at a position where the surface of the object to be treated cannot be heated. Even if the treatment surface of the object to be treated cannot be heated, the treatment device using active oxygen according to the present embodiment can perform the treatment by exposing the treatment surface to the active oxygen in the induced flow. Furthermore, in disinfection treatment using the active oxygen supply device according to the present disclosure, it is possible to eliminate bacteria located at a position where active oxygen can reach. Accordingly, for example, even bacteria located between fibers can be eliminated.

On the other hand, if the heating device is arranged so that it can heat the surface of an object to be treated placed outside the housing through the opening, the undecomposed ozone present in the induced flow can be decomposed in situ on the treatment surface and active oxygen can be generated on the treatment surface. As a result, the degree of treatment and the efficiency of treatment can be further improved.

Furthermore, the distance between the heating device and the surface of the object to be treated may be adjusted depending on the purpose of the treatment. Considering the lifespan of active oxygen contained in the induced flow, the distance is preferably 10 mm or less, and more preferably 4 mm or less, although there is no particular limitation thereto. However, it is not necessary to place the object to be treated such that the treatment surface of the object to be treated is within about 10 mm from the heating device, and the distance between the heating device and the object to be treated is not particularly limited as long as the active oxygen in the induced flow can reach the effective concentration corresponding to the purpose of the treatment, in relation to the heating method and the like.

Also, the amount of ozone generated per unit time in the plasma actuator in a state where the induced flow is not heated is preferably, for example, 15 μg/min or more. More preferably, it is 30 μg/min or more. The upper limit of the amount of ozone generated is not particularly limited, but is, for example, 1000 μg/min or less. That is, the preferred range is from 15 μg/min to 1000 μg/min.

The flow rate of the induced flow may be, for example, a rate at which the generated active oxygen can be actively supplied to the surface region of the object to be treated while maintaining the effective active oxygen concentration or the effective active oxygen amount corresponding to the purpose of the treatment. For example, as described above, the flow rate is about 0.01 m/s to 100 m/s.

As described above, the concentration of ozone in the induced flow generated from the plasma actuator and the flow rate of the induced flow can be controlled by the thicknesses and materials of the electrodes and the dielectric material, the type, amplitude, and frequency of the applied voltage, and the like.

From the viewpoint of lowering the temperature of the plasma actuator 103 and efficiently generating ozone, a shielding member for shielding heat from reaching the plasma actuator may also be provided between the plasma actuator 103 and the heating device 102. A reflective member that reflects heat may also be provided on the heating device side of the shielding member. Examples of the heat-reflecting member include metal tape, metal film, and a metal plate, which contain metals such as aluminum, copper, or stainless steel. When providing a shielding member, it is preferable to provide the shielding member at a position that does not impede the induced flow. Specifically, it is preferable that no shielding member is included on the extension line in the direction along the surface of the dielectric material 201 from the edge of the first electrode 203 of the plasma actuator or in the expulsion direction of the induced flow.

Also, in order to lower the temperature of the plasma actuator 103, the plasma actuator 103 may be provided with a water-cooled heat sink or an air-cooled heat sink.

### <Housing and Opening>

The active oxygen supply device of the present disclosure includes a housing 107 having at least one opening 106, a heating device 102 disposed inside the housing, and a plasma actuator 103.

The opening is not particularly limited as long as the induced flow 105 containing active oxygen generated by the plasma actuator 103 and the heating device 102 flows out of the housing 107. The size of the opening, the position of the opening, and the relative position between the opening and the object to be treated can be selected such that, for example, the generated active oxygen can be actively supplied to the surface region of the object to be treated while maintaining the effective active oxygen concentration or the effective active oxygen amount corresponding to the purpose of the treatment.

Furthermore, it is preferable that the distance between the plasma actuator and the opening is close to the distance between the plasma actuator and the object to be treated in order to more effectively use the active oxygen in the induced flow for the intended treatment. For this reason, it is preferable to arrange the plasma actuator at a position closer to the opening. On the other hand, in order to protect the plasma actuator, it is also preferable to arrange the plasma actuator at a position set back from the opening. For example, it is preferable to arrange the plasma actuator on the inner wall of the housing such that the end of the plasma actuator on the side closer to the opening is located 0.5 mm to 1.5 mm from the edge of the opening of the inner wall of the housing.

The active oxygen supply device of the present disclosure can be used not only for disinfection of an object to be treated, but also for all applications implemented by supplying active oxygen to an object to be treated. For example, the active oxygen supply device of the present disclosure can be used for deodorizing the object to be treated, bleaching the object to be treated, and performing hydrophilization treatment on the surface of the object to be treated.

Also, the treatment device using active oxygen of the present disclosure not only performs treatment for disinfecting an object to be treated, but also can be used for, for example, treatment for deodorizing an object to be treated, treatment for bleaching an object to be treated, surface treatment for hydrophilizing an object to be treated, and the like.

Also, the present disclosure provides a treatment method using active oxygen for treating a surface of an object to be treated with active oxygen, including:
a step of preparing a treatment device using active oxygen;
a step of placing the prepared treatment device using active oxygen and the object to be treated at relative positions where a surface of the object to be treated is exposed when an induced flow containing active oxygen is discharged from an opening; and
a step of treating the surface of the object to be treated with active oxygen by causing the induced flow containing active oxygen to flow out from the opening.

Note that in the present disclosure, “effective active oxygen concentration or effective active oxygen amount” refers to the active oxygen concentration or the active oxygen amount for achieving the purpose of treating the object, such as sterilization, deodorization, bleaching, or hydrophilization, and the effective active oxygen concentration or the effective active oxygen amount can be adjusted as appropriate according to the purpose by using the thicknesses of the electrodes and the dielectric material constituting the plasma actuator, the materials, the type, amplitude, and frequency of the applied voltage, the heating temperature and heating time, the PA incidence angle, and the like.

The active oxygen supply device according to the present disclosure may perform treatment while moving at least one of the active oxygen treatment device and the object to be treated, for example, when the area of the treatment surface of the object to be treated is wider than the opening. The relative movement speed and movement direction between the active oxygen supply device and the object to be treated at this time may be set as appropriate within a range where the treatment surface can be treated to a desired degree, and there is no particular limitation thereto. Similarly, the number of times the object to be treated is treated may be set as appropriate within a range where the treatment surface can be treated to a desired degree.

### EXAMPLES

Hereinafter, the present disclosure will be described in more detail using examples and comparative examples, but the aspects of the present disclosure are not limited thereto.

### Example 1

### 1. Production of Active Oxygen Supply Apparatus

A first electrode was formed by adhering an aluminum foil with a length of 2.5 mm, a width of 15 mm, and a thickness of 100 μm using adhesive tape to a first surface of a glass plate (5 mm long, 18 mm wide (in the depth direction of the page surface in FIG. 1A), and 150 μm thick) serving as a dielectric material. Also, a second electrode was formed by adhering an aluminum foil with a length of 3.0 mm, a width of 15 mm, and a thickness of 100 μm using adhesive tape to a second surface of the glass plate as well so as to be diagonally opposite the aluminum foil adhered to the first surface. Furthermore, the second surface containing the second electrode was covered with polyimide tape. In this way, a plasma actuator was produced by providing the first electrode and the second electrode so as to overlap with each other over a width of 200 μm with a dielectric material (glass plate) interposed therebetween. Two of these plasma actuators were prepared.

Next, as the housing 107 of the active oxygen supply device 101, a case whose cross-sectional shape in the shorter direction (cross-section taken along line A-A′ in FIG. 1B) is a substantially trapezoidal shape shown in FIG. 1A, and that is made of polyether ether ketone and has a height of 25 mm, a width of 20 mm, a length of 170 mm, and a thickness of 2 mm was prepared. FIG. 1B is a diagram showing the case as viewed from the opening 106. The case had a rectangular opening 106 with a width of 7 mm and a length of 15 mm that is symmetrical about a center of a longitudinal length of 170 mm (dashed line A-A′ in FIG. 1B).

Next, the two previously produced plasma actuators 103 were fixed to the oblique side portions of the housing 107 in FIG. 1A. Specifically, regarding the plasma actuators 103, the angle θ (same value as the above-mentioned PA incidence angle) formed by the intersection between the extension line 201-1-1 in the direction along the exposed portion 201-1 of the first surface of the dielectric material 201 and the treatment surface 104-1 of the object to be processed was 45°. Also, the attachment positions of the plasma actuators 103 (dashed line portions in FIG. 1B) as viewed from the bottom of the housing (FIG. 1B) are fixed such that the center line of the housing with a length of 170 mm (one-dot broken line), the center line of the opening, and the centers of the plasma actuators coincide with each other.

Furthermore, a cartridge heater 102 (product name: HLC0061, manufactured by Hakko Electric Co., Ltd.) was arranged inside the housing. The cartridge heater 102 was arranged such that the distance (reference numeral 403 in FIG. 4) between the cartridge heater 102 and the exposed portion 201-1 of the first surface of the dielectric material 201 of the plasma actuator was 2 mm and the distance (reference numeral 401 in FIG. 4) between the heating device and the surface on the side of the flat plate facing the heating device when the flat plate was in contact with the opening 106 of the housing 107 was 3 mm. In this way, the active oxygen supply device (treatment device using active oxygen) according to this example was produced.

A K-type thermocouple (product name: HTK3501, manufactured by Hakko Electric Co., Ltd.) was placed at the position of the opening 106 that serves as the supply port for active oxygen in this active oxygen supply device 101, and the cartridge heater 102 was controlled such that the temperature measured at this location was 120° C.

Subsequently, in order to calculate the amount of ozone generated from the plasma actuator 103, the active oxygen supply device 101 was placed in a closed container (not shown) with a volume of 1 liter. The closed container was provided with a hole that could be sealed with a rubber stopper, and the gas inside could be sucked through the hole with a syringe. Then, one minute after applying an AC voltage having a sinusoidal waveform with an amplitude of 2.4 kV and a frequency of 80 kHz to the plasma actuator 103 without operating the cartridge heater, 100 ml of the gas in the closed container was collected. The collected gas was sucked into an ozone detection tube (product name: 182SB, manufactured by Komei Rikagaku Kogyo Co., Ltd.), and the measured ozone concentration (PPM) contained in the induced flow from the plasma actuator 103 was measured. Using the value of the measured ozone concentration, the amount of ozone generated per unit time was determined with the following formula.

$$Amount of ozone generated per unit time ( \frac{mg}{min} ) = \qquad [ Math . 1 ]$$

$$Measured ozone concentration ( PPM ) * \frac{Ozone molecular weight 48}{22.4} * \frac{\frac{273}{273 + Room temperature ( ° C . )}}{10000} * \frac{Gas inside closed container ( L )}{Collected gas ( L )} = Measured ozone concentration ( PPM ) * 48 / 22.4 * 273 / ( 273 + 25 ) / 10000 * 0.1 / 1$$

As a result, the amount of ozone generated per unit time was 39 μg/min.

Finally, the amount of ozone generated when both the plasma actuator 103 and the cartridge heater 102 were in operation was measured. The operating conditions for the plasma actuator 103 are such that 39 μg/min of ozone is generated when only the plasma actuator 103 is operated. Also, the operating conditions for the cartridge heater 102 are such that when only the cartridge heater 102 is operated, the temperature becomes 120° C. As a result, the amount of ozone generated when both the plasma actuator 103 and the cartridge heater 102 were operating was 21 μg/min. It is thought that the decrease of 18 μg/min from 39 μg/min is the amount of ozone that was converted to active oxygen.

### 2-1. Active Oxygen Detection Test (Methylene Blue Absorbance)

Decolorization of methylene blue was used to check whether or not there was active oxygen in the induced flow flowing out from the opening (see Masanobu WAKASA et al., “Magnetic Field Effect on the Photocatalytic Reaction with TiO2 Semiconductor Film”, Journal of The Society of Photographic Science and Technology of Japan, 69, 4, 271-275 (2006)). Methylene blue is a crystalline powder with a blue luster and is soluble in water and ethanol, and therefore it is used in solution as a dye or indicator. Also, methylene blue reacts with active oxygen, decomposes, and loses its blue color. For this reason, the decolorization of methylene blue (loss of blue color) can be used to check whether or not there is active oxygen in the induced flow. Specifically, the following operations were performed. Methylene blue (manufactured by Kanto Kagaku, special grade) and distilled water were mixed to prepare a 0.01% methylene blue aqueous solution. 15 ml of the methylene blue aqueous solution was placed in a petri dish (AB4000 manufactured by Eiken Kagaku, cylindrical shape, 88 mm diameter). Then, the liquid surface of the methylene blue aqueous solution in the petri dish was regarded as the treatment surface 104-1 of the object to be treated, and the active oxygen supply device 101 was arranged above the petri dish such that the distance 405 in FIG. 4 was 3 mm.

Next, an AC voltage having a sinusoidal waveform with an amplitude of 2.4 kV and a frequency of 80 kHz was applied between the first electrode and the second electrode of the plasma actuator of the active oxygen supply device, the cartridge heater was activated, and the induced energy flowing out from the opening was supplied to the liquid surface for 150 minutes.

After the induced flow irradiation, the aqueous methylene blue solution was transferred from the petri dish to a cell, and changes in the amount of light absorption of methylene blue were measured using a spectrophotometer (product name: V-570; manufactured by JASCO Corporation). Since methylene blue has strong absorption at a wavelength of 664 nm, the degree of decolorization of methylene blue can be calculated from the change in absorbance at this wavelength. In this test, first, only distilled water was placed in a reference cell, and the induced flow was measured by placing 0.01% methylene blue aqueous solution before irradiation in the sample cell, and the absorbance was 2.32 Abs. On the other hand, the absorbance of the methylene blue aqueous solution after induced flow irradiation was 0.07 Abs. Thus, the rate of decrease in absorbance was ((2.32−0.07)/2.32)×100-97.0%.

### 2-2. Treatment (Disinfection) Test

The active oxygen supply device 101 according to this example was used to carry out an E. coli disinfection test in the following procedure. Note that all the instruments used in this disinfection test were subjected to high-pressure steam sterilization using an autoclave. In addition, this sterilization test was conducted in a clean bench.

First, Escherichia coli (product name: KWIK-STIK (Escherichia coli ATCC8739), manufactured by Microbiologics) was placed in an Erlenmeyer flask containing LB medium (2 g of tryptone, 1 g of yeast extract, 1 g of sodium chloride, mixed with distilled water to make 200 ml), and was shake-cultured at 80 rpm at a temperature of 37° C. for 48 hours. After culturing, the E. coli solution was 9.2×10⁹ (CFU/ml).

Sample No. 1 was prepared by using a micropipette to drip 0.010 ml of this cultured bacterial solution onto a qualitative filter paper (product number: No. 5C, manufactured by Advantech) with a length of 3 cm and a width of 1 cm. Also, the bacterial solution was only dripped onto one surface of the filter paper. Sample No. 2 was prepared in the same manner.

Next, sample No. 1 was immersed in a test tube containing 10 ml of buffer solution (product name: Gibco PBS; Thermo Fisher Scientific) for 1 hour. Note that in order to prevent the bacterial solution on the filter paper from drying out, the time from dripping the bacterial liquid onto the filter paper to immersion in the buffer solution was set to 60 seconds.

Next, 1 ml of the buffer solution in which sample No. 1 was immersed (hereinafter referred to as “1/1 solution”) was placed into a test tube containing 9 ml of buffer solution to prepare a diluted solution (hereinafter referred to as “1/10 diluted solution”). A 1/100 diluted solution, a 1/1000 diluted solution, and a 1/10000 diluted solution were prepared in the same manner except that the dilution ratio with the buffer solution was changed.

Next, 0.050 ml of the 1/1 solution was collected and smeared on a stamp medium (Petan Check 25 PT1025 manufactured by Eiken Kasei Co., Ltd.). This operation was repeated to create two stamp media on which 1/1 solution was smeared. The two stamp media were placed in a constant temperature bath (product name: IS600; manufactured by Yamato Scientific Co., Ltd.) and cultured at a temperature of 37° C. for 24 hours. The number of colonies generated on the two stamp media was counted, and the average value was calculated.

For the 1/10 diluted solution, 1/100 diluted solution, 1/1000 diluted solution, and 1/10000 diluted solution, two smeared stamp media were prepared and cultured for each diluted solution in the same manner as above. Then, the number of colonies generated in each stamp medium for each diluted solution was counted, and the average value was calculated. The results are shown in Table 1-1.

TABLE 1-1

| | | Sample No. 1 (blank) |
|---|---|---|
| 1/1 | Solution | >100 |
| 1/10 | Solution | >100 |
| 1/100 | Solution | 54 |
| 1/1000 | Solution | 4 |
| 1/10000 | Solution | 0 |

From the results in Table 1-1 above, it was found that the number of colonies when the 1/100 diluted solution was cultured was 54, and accordingly, the number of bacteria present in 0.050 ml of the 1/1 solution of sample No. 1 was 54×10²=5400 (CFU).

Next, the following operation was performed on sample No. 2.

A recess with a length of 3.5 cm, a width of 1.5 cm, and a thickness of 1.2 mm was provided in the center of a plastic flat plate with a length of 30 cm, a width of 30 cm, and a thickness of 5 mm. A filter paper with a length of 3.5 cm and a width of 1.5 cm was placed in the recess. Sample No. 2 was placed on this filter paper so that the surface onto which the bacterial solution was dripped faced the filter paper placed at the bottom of the recess. Then, the active oxygen supply device was arranged on the upper surface of the plastic plate such that the longitudinal center of the opening thereof coincided with the longitudinal center of the recess, and the widthwise center of the opening coincided with the center in the short direction of the recess, and such that the distance 405 shown in FIG. 4 (distance from the opening side distal end of the plasma actuator to the surface of the filter paper facing the cartridge heater) was 1.4 mm. Note that since the depth of the recess was 2 mm and the thickness of the filter paper was 0.2 mm, the bacterial liquid adhesion surface of each sample did not come into direct contact with the opening of the active oxygen supply device. Next, an AC voltage having a sinusoidal waveform with an amplitude of 2.4 kVpp and a frequency of 80 kHz was applied between both electrodes of the active oxygen supply device, and the cartridge heater was activated to supply an induced flow toward the filter paper. The supply time (processing time) was 10 seconds. Note that the cartridge heater was adjusted so that the temperature measured at the surface of the filter paper facing the cartridge heater was 120° C. without turning on the plasma actuator.

Also, in order to prevent the filter paper onto which the bacterial solution was dripped from drying out as much as possible during the treatment process using the active oxygen supply device, the time from dripping the bacterial solution onto the filter paper to immersion in the buffer solution was set to 60 seconds.

The treated sample No. 2 was immersed for 1 hour in a test tube containing 10 ml of buffer solution (product name: Gibco PBS; Thermo Fisher Scientific) together with a filter paper placed at the bottom of the recess. Next, 1 ml of the buffer solution after immersion (hereinafter referred to as “1/1 solution”) was placed in a test tube containing 9 ml of buffer solution to prepare a diluted solution (1/10 diluted solution). A 1/100 diluted solution, a 1/1000 diluted solution, and a 1/10000 diluted solution were prepared in the same manner except that the dilution ratio with the buffer solution was changed.

Next, 0.050 ml of the 1/1 solution was collected and smeared on a stamp medium (product name: Petan Check 25 PT1025, manufactured by Eiken Kasei Co., Ltd.). This operation was repeated to create two stamp media on which 1/1 solution was smeared. A total of two stamp media were placed in a constant temperature bath (product name: IS600; manufactured by Yamato Scientific Co., Ltd.) and cultured at a temperature of 37° C. for 24 hours. The number of colonies generated for each stamp medium for the 1/1 solution was counted, and the average value was calculated. For the 1/10 diluted solution, 1/100 diluted solution, 1/1000 diluted solution, and 1/10000 diluted solution, two smeared stamp media were prepared and cultured for each diluted solution in the same manner as above. Then, the number of colonies generated in each stamp medium for each diluted solution was counted, and the average value was calculated. The results are shown in Table 1-2 below.

TABLE 1-2

| | | Sample No. 2 |
|---|---|---|
| 1/1 | Solution | 11 |
| 1/10 | Solution | 1 |
| 1/100 | Solution | 0 |
| 1/1000 | Solution | 0 |
| 1/10000 | Solution | 0 |

As shown in Table 1-1, the number of bacteria in 0.050 ml of 1/1 solution of sample No. 1 that was not treated with the active oxygen supply device was 5400 (CFU), but the number of bacteria in 0.050 ml of 1/1 solution of Sample No. 2 after treatment was 11 (CFU). From this, it was found that 99.796% ((5400-11/5400)×100) of bacteria was eliminated by the 2-second treatment using the active oxygen supply device according to this example.

### Examples 2 and 3

A decolorization test of the ozone methylene blue aqueous solution and a disinfection test were performed in the same manner as in Example 1, except that the strength of the cartridge heater was adjusted so that the temperature on the surface of the filter paper facing the cartridge heater during treatment was 100° C. or 80° C.

### Examples 4 and 5

A plasma actuator according to Example 4 or Example 5 was produced in the same manner as the plasma actuator according to Example 1, except that the thickness of the dielectric material in the plasma actuator according to Example 1 was changed to 200 μm or 250 μm.

An active oxygen supply device was produced, and measurement of the amount of ozone generated, a decolorization test of methylene blue aqueous solution, and a disinfection test were performed in the same manner as in Example 1, except that these plasma actuators were used.

### Example 6

A plasma actuator according to Example 6 was produced in the same manner as the plasma actuator according to Example 1, except that the material of the dielectric material of the plasma actuator according to Example 1 was changed to polyimide. An active oxygen supply device was produced, measurement of the amount of ozone generated, a decolorization test of methylene blue aqueous solution, and a disinfection test were performed in the same manner as in Example 1, except that this plasma actuator was used.

### Comparative Examples 1 and 2

Comparative Examples 1 and 2 used the same conditions as Example 1, except that they had the following configurations.

Comparative Example 1: Voltage was applied to the plasma actuator and no heating device was used.

Comparative Example 2: A heating device was used without applying voltage to the plasma actuator.

The evaluation results of Examples 1 to 6 and Comparative Examples 1 and 2 are shown in Table 2.

TABLE 2

| | | | | Temperature of | | Percentage of | Disinfection test | |
|---|---|---|---|---|---|---|---|---|
| | | | | surface region | Amount of | decrease in | Disinfection performance | |
| | | Dielectric | | of treatment | ozone | absorbance of | determination | |
| | | thickness | Dielectric | surface | generated | methylene | (number of | Disinfection |
| | | (μm) | material | (° C.) | (μg/min) | blue (%) | colonies CFU) | rate |
| Example | 1 | 150 | Glass | 120 | 39 | 97.0% | 11 | 99.80% |
| | 2 | 150 | Glass | 100 | 39 | 92.3% | 45 | 99.17% |
| | 3 | 150 | Glass | 80 | 39 | 84.9% | 120 | 97.78% |
| | 4 | 200 | Glass | 120 | 34 | 93.1% | 38 | 99.30% |
| | 5 | 250 | Glass | 120 | 10 | 87.0% | 78 | 98.56% |
| | 6 | 150 | Polyimide | 120 | 38 | 95.8% | 17 | 99.69% |
| Comparative | 1 | 150 | Glass | 25 | 39 | 39.4% | 4900 | 9.26% |
| Example | 2 | 150 | Glass | 120 | 0 | 5.4% | 1300 | 75.93% |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An active oxygen supply device comprising a plasma actuator and a heating device inside a housing having at least one opening,
wherein the plasma actuator is formed by stacking a first electrode, a dielectric material, and a second electrode in the stated order,
the first electrode is an exposed electrode provided on a first surface that is one surface of the dielectric material,
when a voltage is applied between the first electrode and the second electrode, the plasma actuator generates a dielectric-barrier discharge from the first electrode to the second electrode and expels an induced flow containing ozone in a first direction, which is one direction along a surface of the dielectric material from the first electrode,
the heating device heats the induced flow containing the ozone to generate active oxygen in the induced flow, and the induced flow becomes an induced flow containing the active oxygen, and
the plasma actuator and the heating device are arranged such that the induced flow containing the active oxygen flows out of the housing through the opening.

2. The active oxygen supply device according to claim 1,
wherein in a view of a cross-section of the plasma actuator taken in a thickness direction,
the first electrode and the second electrode are arranged diagonally opposite each other in the thickness direction of the plasma actuator with the dielectric material interposed therebetween,
the first electrode is provided so as to cover a part of the first surface of the dielectric material,
the first surface has an exposed portion that is not covered by the first electrode,
in a view through the plasma actuator from the first electrode side, there is an overlap between at least a part of the exposed portion and the second electrode, and
the induced flow containing the ozone is expelled along the exposed portion of the dielectric material overlapping with the second electrode, from an edge on a side of the first electrode in the first direction in the cross-section taken in the thickness direction.

3. The active oxygen supply device according to claim 1,
wherein in a cross-section of the plasma actuator taken in the thickness direction, when an edge on a side of the first electrode in the first direction is an edge A and an edge on a side of the second electrode in a second direction opposite to the first direction is an edge B,
the edge B is located in the second direction relative to the edge A.

4. The active oxygen supply device according to claim 1,
wherein in a view through the plasma actuator from the first electrode side, when an edge on a side of the first electrode in the first direction is an edge A and an edge on a side of the second electrode in a second direction opposite to the first direction is an edge B,
the edge B is located in the second direction relative to the edge A.

5. The active oxygen supply device according to claim 1,
wherein in a cross-section of the plasma actuator taken in the thickness direction, when an edge on a side of the first electrode in the first direction is an edge A and an edge on a side of the second electrode in a second direction opposite to the first direction is an edge B,
the edge B is located in the first direction relative to the edge A.

6. The active oxygen supply device according to claim 1,
wherein in a view through the plasma actuator from the first electrode side, when an edge on a side of the first electrode in the first direction is an edge A and an edge on a side of the second electrode in a second direction opposite to the first direction is an edge B,
the edge B is located in the first direction relative to the edge A.

7. The active oxygen supply device according to claim 1,
wherein in a view through the plasma actuator from the first electrode side, when an edge on a side of the first electrode in the first direction is an edge A and an edge on a side of the second electrode in a second direction opposite to the first direction is an edge B,
the edge A and the edge B coincide with each other in the thickness direction of the dielectric material.

8. The active oxygen supply device according to claim 1, wherein the amount of ozone generated per unit time while the induced flow is not heated in the plasma actuator is 15 μg/min or more.

9. The active oxygen supply device according to claim 1, wherein when the opening of the active oxygen supply device is directed vertically downward, a narrow angle θ formed by a horizontal plane and an extension line in a direction along the exposed portion of the first surface of the dielectric material from the edge of the first electrode of the plasma actuator is 0° to 90°.

10. The active oxygen supply device according to claim 1, wherein a distance between the heating device and the plasma actuator is 10 mm or less.

11. The active oxygen supply device according to claim 1, wherein the heating device is arranged such that an object to be treated placed outside of the housing can be heated via the opening.

12. A treatment device using active oxygen, configured to treat a surface of an object to be treated with active oxygen, the treatment device comprising a plasma actuator and a heating device inside a housing having at least one opening,
wherein the plasma actuator is formed by stacking a first electrode, a dielectric material, and a second electrode in the stated order,
the first electrode is an exposed electrode provided on a first surface that is one surface of the dielectric material,
when a voltage is applied between the first electrode and the second electrode, the plasma actuator generates a dielectric-barrier discharge from the first electrode to the second electrode and expels an induced flow containing ozone in a first direction, which is one direction along a surface of the dielectric material from the first electrode,
the heating device heats the induced flow containing the ozone to generate active oxygen in the induced flow, and the induced flow becomes an induced flow containing the active oxygen, and
the plasma actuator and the heating device are arranged such that the induced flow containing the active oxygen flows out of the housing through the opening.

13. The treatment device using active oxygen according to claim 12, wherein the heating device is arranged such that a surface of the object to be treated can be heated.

14. A treatment method using active oxygen, for treating a surface of an object to be treated with active oxygen, the method comprising
a step of preparing a treatment device using active oxygen,
wherein the treatment device using active oxygen includes a plasma actuator and a heating device in a housing having at least one opening,
the plasma actuator is formed by stacking a first electrode, a dielectric material, and a second electrode in the stated order,
the first electrode is an exposed electrode provided on a first surface that is one surface of the dielectric material,
when a voltage is applied between the first electrode and the second electrode, the plasma actuator generates a dielectric-barrier discharge from the first electrode to the second electrode and expels an induced flow containing ozone in a first direction, which is one direction along a surface of the dielectric material from the first electrode,
the heating device heats the induced flow containing the ozone to generate active oxygen in the induced flow, and the induced flow becomes an induced flow containing the active oxygen,
the plasma actuator and the heating device are arranged such that the induced flow containing the active oxygen flows out of the housing through the opening, and the method further comprises:
a step of placing the prepared treatment device using active oxygen and the object to be treated at relative positions at which a surface of the object to be treated is exposed when the induced flow containing the active oxygen flows out through the opening; and
a step of allowing the induced flow containing the active oxygen to flow out through the opening to treat the surface of the object to be treated with active oxygen.

15. The treatment method using active oxygen according to claim 14, wherein the distance between the heating device and the surface of the object to be treated is 10 mm or less.
